(19) United States
(12) Patent Application Publication
(10) Pub. No.: US 2010/0030231 A1
(43) Pub. Date: Feb. 4, 2010

(54) SURGICAL SYSTEM AND METHOD

(76) Inventors: Ian Revie, Boroughbridge (GB); Yaacov Nitzan, Hertzeliya (IL); Mike Slomczykowski, Leeds (GB); Joerg Haechler, Baar (CH)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/916,168

(22) PCT Filed: Jun. 1, 2006
(86) PCT No.: PCT/GB06/01985

§ 371 Date: May 18, 2009

(30) Foreign Application Priority Data
Jun. 2, 2005 (EP) 05253546.5

(51) Int. Cl. A61B 19/00; G06K 9/00

(52) U.S. Cl. 606/130; 382/128

(57) ABSTRACT

Methods, systems and data processing apparatus and methods are described for use in planning and carrying out a surgical procedure on a joint of a subject. The joint includes a first component and a second component, the first and second components being relatively movable. Joint data describing the configuration of the joint is captured while the joint is in a functional state wherein the joint is undergoing its usual load bearing functional behaviour. The joint data is analyzed to determine joint component orientation data specifying the relative orientation of the first and second components. The orientation of prosthetic implants to be used in the surgical procedure to recreate the joint is planned using the joint component orientation data. The planned orientation of the prosthetic implants improves the recreation of the functional state of the joint.

The present invention relates to surgical systems and methods, and in particular to systems and methods allowing a surgeon to more realistically recreate the functional performance of a part of a patient.

A surgical procedure is generally carried out with the patient recumbent on a table in an operating theatre. The patient's skeleton and joints are differently loaded by the action of gravity on the patient's body mass in a recumbent position compared to when the patient is in other positions, such as standing or sitting. For example, when standing, the patient's leg joints, such as the hip or knee, are in a loaded state in which the weight of the patient's upper body is supported. Further, the joints are exposed to different dynamic loads as a patient carries out dynamic activities, such as walking or moving from a standing to a sitting position. It is these functional behaviours of the joints that a surgeon should be aiming to reproduce during surgery.

However, when a patient is recumbent, body parts are not exposed to the same loads as when the patient's body is in other positions. Further, during surgery, it is often necessary to cut, excise, remove or otherwise disconnect soft tissue from around the body part, such as muscles or ligaments. Hence, the soft support structures of, for example, a joint, and which also help to define the correct functional configuration of the joint, can be absent or in a different state, so that the joint presented to the surgeon during surgery has a different configuration to the intended functional configuration that it is intended to recreate. Therefore the surgical site itself may not provide useful guidance as to how to reconstruct the functional configuration of the joint.

Therefore, it would be advantageous to be able to more reliably recreate the functional configuration of a joint in a surgical procedure.

The present invention uses data derived from the patients body in its functional state in order to correct or adapt the surgical procedure so as to more accurately reproduce the functional configuration of a joint and therefore the functional behaviour of the patient's body.

According to the present invention, there is provided a method for planning a joint replacement surgical procedure, in which at least one part of the joint is to be replaced by an implant. The method can include planning the position and/or orientation of the implant based on the position and/or orientation of the part of the joint while the joint is in a functional state.

Hence, by planning the implant position and/or orientation using the position and/or orientation of the part of the joint in its functional state, i.e. while performing its in use load bearing activity, the functional configuration of the joint can be more accurately reproduced when the implant is implanted at the planned position.

According to a further aspect of the present invention, there is provided a method for planning a surgical procedure to be carried out on a joint of a subject. The method can comprise capturing joint data describing the configuration of the joint while the joint is in a functional state. The joint data can be analyzed or processed to determine joint component orientation data specifying the orientation of the component or components of the joint. The orientation of a prosthetic implant or implants to be used in the surgical procedure to recreate the joint can be planned using the joint component orientation data.

The planned orientation of the prosthetic implant or implants can, or can be intended to, substantially recreate the functional state of the joint.

The planned orientation of the prosthetic implant or implants can be defined in relation to the subject's functional state.

The joint data can be captured in various different ways. The joint data can have been captured using imaging. At least one image of the joint in the functional state can be captured. Preferably, at least two images of the joint in the functional state are captured. Preferably the at least two images are from different directions and preferably substantially perpendicular directions.

The joint data can have been captured by tracking the position and/or orientation of the joint component or components in the functional state. Trackable markers attached to the or each joint component can be used to allow the position and/or orientation of the or each component to be tracked. A single or multiple trackable markers can be attached to each joint component in order to allow the orientation of the or each component, or part thereof, to be tracked.

Analyzing the joint data can include determining the orientation of the or each joint component relative to the or each image of the or each joint component.

Analyzing the joint data can include determining the orientation of the or each joint component relative to a reference direction. Any reference direction having a known direction or orientation can be used. Preferably the reference direction is the direction of gravity at the location where the joint data was captured.

The method can further comprise determining the orientation of a reference direction when the joint data is captured. Preferably the reference direction is the direction of gravity at the location where the joint data was captured.

The method can further comprise determining the orientation of the or each joint component relative to the reference direction.

The method can further comprise determining the orientation of a first component relative to the local anatomy of the subject and/or determining the orientation of a second component relative to the local anatomy. The first component and/or the second component can be a part of a bone. The first component can be a proximal part of the femur and the local anatomy can be an axis or axes of the femur. The second component can be the acetabulum and the local anatomy can be a plane of planes of the pelvis.

Planning can include displaying at least one image of the joint. Preferably the image or images include all of the components of the joint. Preferably images of the joint from different directions are displayed. The or each image can be a captured image or an image derived from a captured image or images. Planning can include displaying an image of an implant or implants.

Planning can includes displaying a visual indication of the orientation of the or each component. The orientation of the component or components can be displayed relative to a reference direction and/or a local anatomical direction or directions.

According to a further aspect of the invention, there is provided a method for carrying out a computer aided surgery procedure on a joint of a subject. The position and orientation of a prosthetic implant or implant to be used in the surgical procedure to recreate the joint can be planned according to any of the methods of the preceding aspect of the invention. A visual indication of the planned position and orientation of the prosthetic implant or implants relative to the subject's joint can be displayed. Further, or alternatively, a visual indication of the real time position and orientation of the prosthetic implant or implants relative to the subject's joint can be displayed. Hence, the implantation of the prosthetic implants can be guided so as to recreate the functional joint.

According to a further aspect of the invention, there is provided a method for assessing a surgical procedure carried out to replace a joint, or part of a joint, of a subject. Before surgery, joint data describing the configuration of the joint while the joint is in a functional state can be captured. Joint component orientation data specifying the orientation of the component, or components, of the joint can be determined. After surgery, joint data describing the configuration of the joint while the joint is in the same functional state can be captured. Joint component orientation data specifying the orientation of the component or components can be determined. The before surgery joint orientation data and after surgery joint orientation data can be compared to assess how well the joint has been recreated.

According to a further aspect of the invention, there is provided a data processing apparatus for planning a surgical procedure to be carried out on a joint of a subject. The apparatus can include at least one data processor configurable by computer program code instructions. The instructions can cause the data processor to capture joint data describing the configuration of the joint while the joint is in a functional state. The joint data can be analyzed to determine joint component orientation data specifying the orientation of the component or components of the joint. The orientation of prosthetic implants to be used in the surgical procedure to recreate the joint can be planned using the joint component orientation data. The planned orientation of the prosthetic implants can, or be intended to, substantially recreate the functional state of the joint.

According to a further aspect of the invention, there is provided a system for planning a surgical procedure to be carried out on a joint of a subject. The system can include data processing apparatus according to the preceding aspect of the invention. The system can include an image capturing device for capturing at least one image of the joint of the subject in the functional state. Additionally, or alternatively, the system can include a tracking device for tracking and determining the position and orientation of markers attachable to the subject and/or parts of the system.

According to a further aspect of the invention, there is provided a data processing apparatus for guiding a computer aided surgery procedure being carried out on a joint of a subject. The apparatus can include a data processing device configurable by computer program code instructions. The instructions can cause the data processing device to display a visual indication of the planned position and/or orientation of the prosthetic implant or implants relative to the subject's joint. The planned position and/or orientation can be derived from planning data created from joint component orientation data specifying the orientation of a component or components of the joint which has been derived from captured joint data describing the configuration of the joint while the joint is in a functional state. A visual indication of the real time position and/or orientation of the implant or implants relative to the subject's joint can be displayed. The implantation of the implant or implants can be guided so as to recreate the functional joint.

According to a further aspect of the invention there is provided a data processing apparatus for assessing a surgical procedure carried out to replace a joint of a subject. The apparatus can comprise a data processing device configurable by computer program code instructions. The instructions can cause the data processing device to obtain pre-surgery joint component orientation data specifying the orientation of a component or components of the joint derived from captured joint data describing the configuration of the joint in a functional state. Post-surgery joint component orientation data specifying the orientation of the component or components derived from captured joint data describing the configuration of the joint in the same functional state can be obtained. A comparison of the pre-surgery joint orientation data and post-surgery joint orientation data can be output to allow assessment of how well the joint has been recreated.

According to a further aspect of the invention, there is provided a method for planning a surgical procedure to be carried out on a joint of a subject, the joint including a first component and/or a second component. The orientation of the first and/or second component can be determined while the joint is in a functional state in which the joint is undergoing its usual load bearing functional behaviour. The orientation of a prosthetic implant or implants to be used in the surgical procedure to recreate the joint can be planned using the orientation of the joint components, and/or their relative orientation. The planned orientation of the prosthetic implants can, or can be intended to, substantially recreate the functional state of the joint.

The joint can include a first and a second components and the first and second components can be relatively movable,

The method can further comprise determining the relative orientation of at least one of the joint components and a reference direction when the joint is in its functional state. The relative orientation can be used during planning the orientation of prosthetic implants to recreate the functional state of the joint.

At least one image of the joint component or components can be captured prior to planning. At least one image of the joint component or components can be captured after the surgical procedure.

According to a further aspect of the invention, there is provided a method for carrying out a surgical procedure on a joint of a subject. The method can comprise planning the orientation of a prosthetic implant or implants according to the preceding method aspect of the invention. The planned position and/or orientation of the prosthetic implant ort implants can be used to guide the implantation of the actual prosthetic implant or implants during surgery so as to recreate the functional state of the joint.

According to a further aspect of the invention, there is provided a method for assessing a surgical procedure carried out on a joint of a subject. The method can comprise pre-operatively determining the orientation of a first and/or second component of the joint while the joint is in a functional state. Post-operatively, the orientation of the first and/or second component can be determined while the joint is in the same functional state. The surgical procedure can be assessed based on the pre and post operative orientation of the joint component or components, and/or their relative orientation.

The method can further comprise pre-operatively determining the relative orientation of the first and/or second joints and a reference direction. The relative orientation of the same first and/or second joints and the reference direction can be determined post-operatively. The pre- and post-operative relative orientations of the first and/or second joint component and the reference direction can be used in the assessment of the surgical procedure.

According to a further aspect of the invention, there is provided computer program code comprising instructions which can be carried out by a data processing devices to provide the various method, apparatus or system aspects of the invention. A computer program product comprising a computer readable medium, or media, bearing such computer program code is also provided.

An embodiment of the invention will now be described, by way of example only, and with reference to the accompanying drawings, in which:

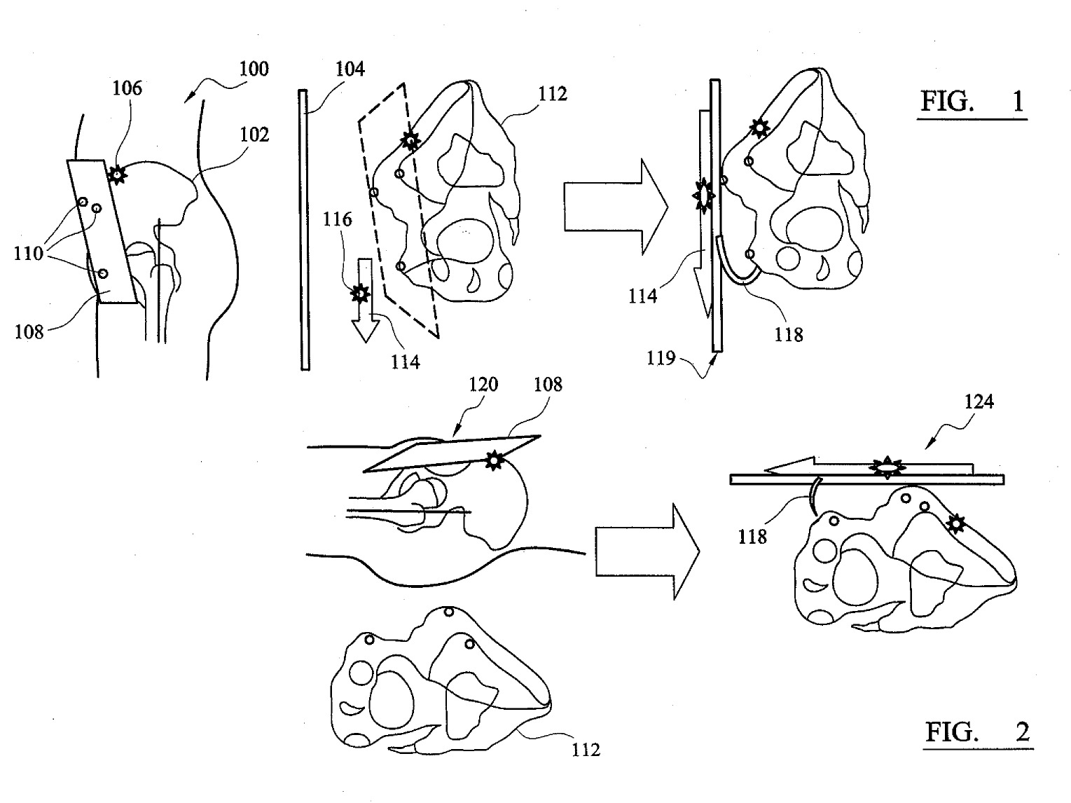

FIG. 1 shows a schematic representation of the pelvis of a patient illustrating the capture of orientation data and reference direction as part of the method of the invention;

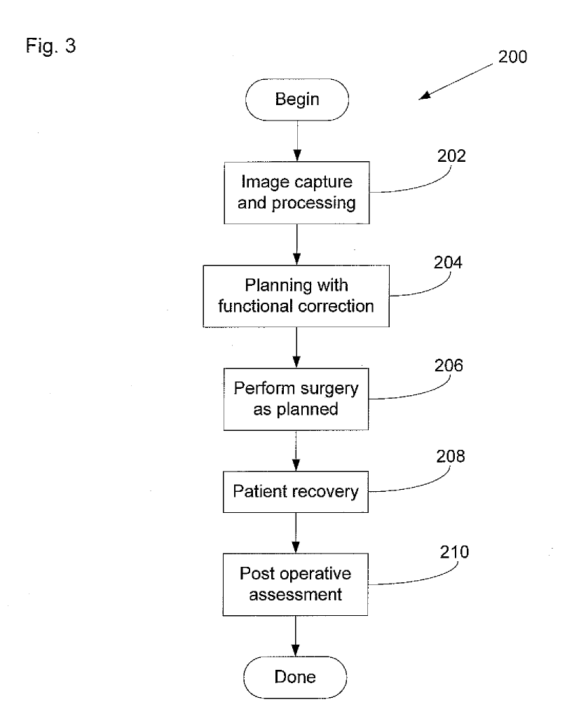

FIG. 2 shows as schematic representation of the pelvis shown in FIG. 1 illustrating the use of the captured orientation data as part of a surgical procedure;

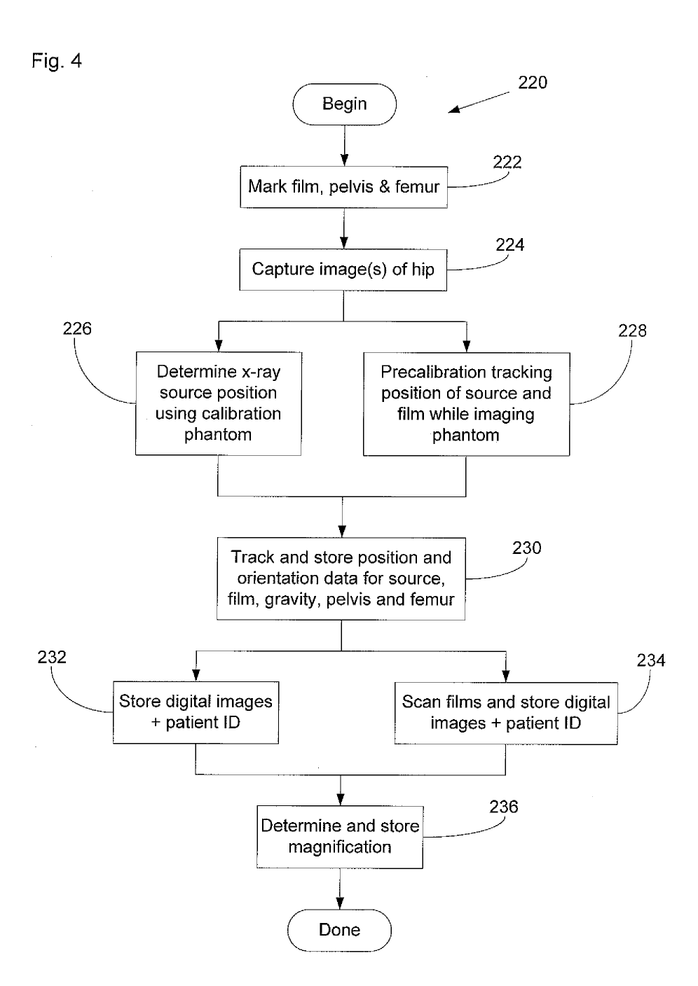

FIG. 3 shows a flow chart illustrating an overall surgical method according to the invention, and including steps each being according to the invention;

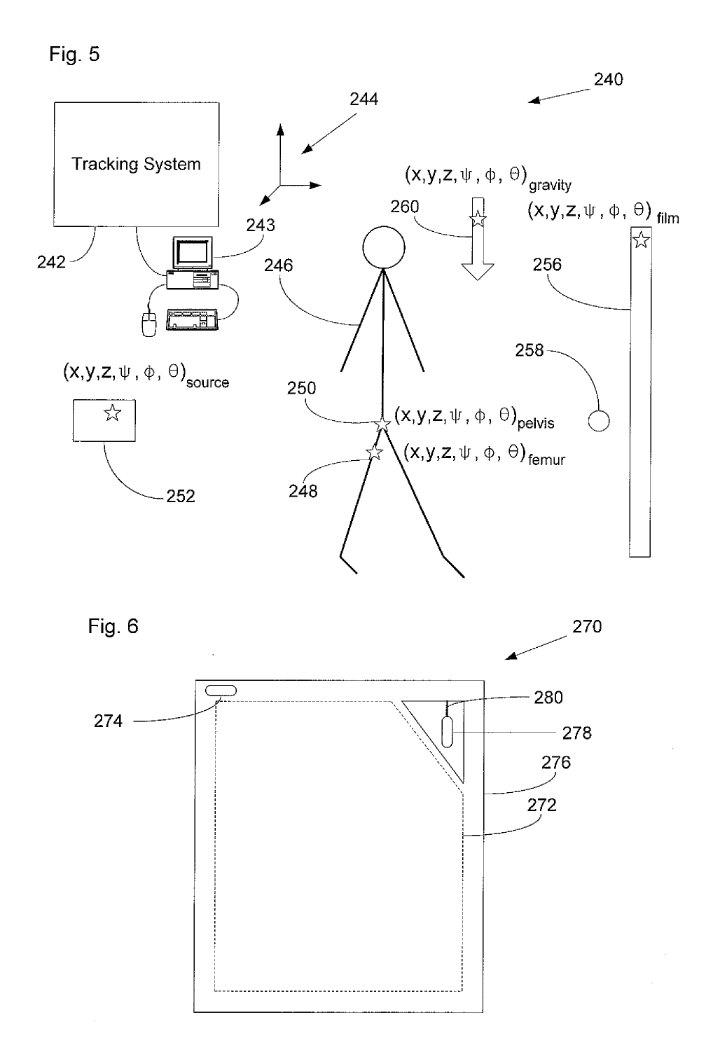

FIG. 4 shows a flow chart illustrating a method for capturing images being a part of the method illustrated in FIG. 3;

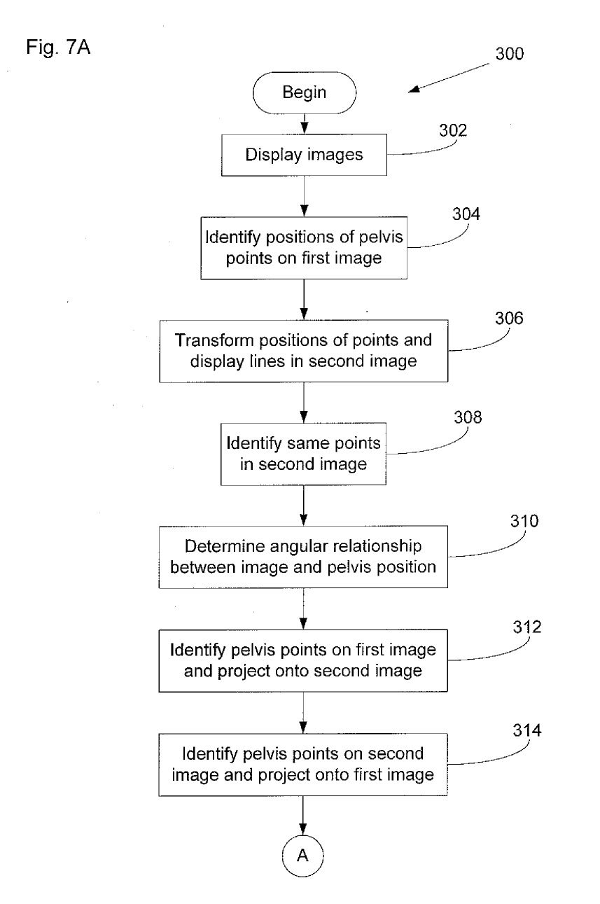

FIG. 5 shows an image capturing system used in the method illustrated by FIG. 4;

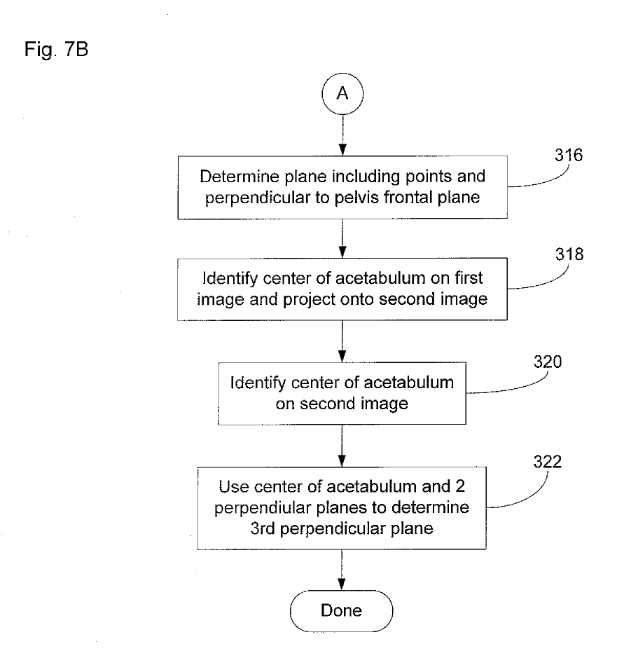

FIG. 6 shows a schematic diagram of a embodiment of an X-ray cassette part of the system shown in FIG. 5;

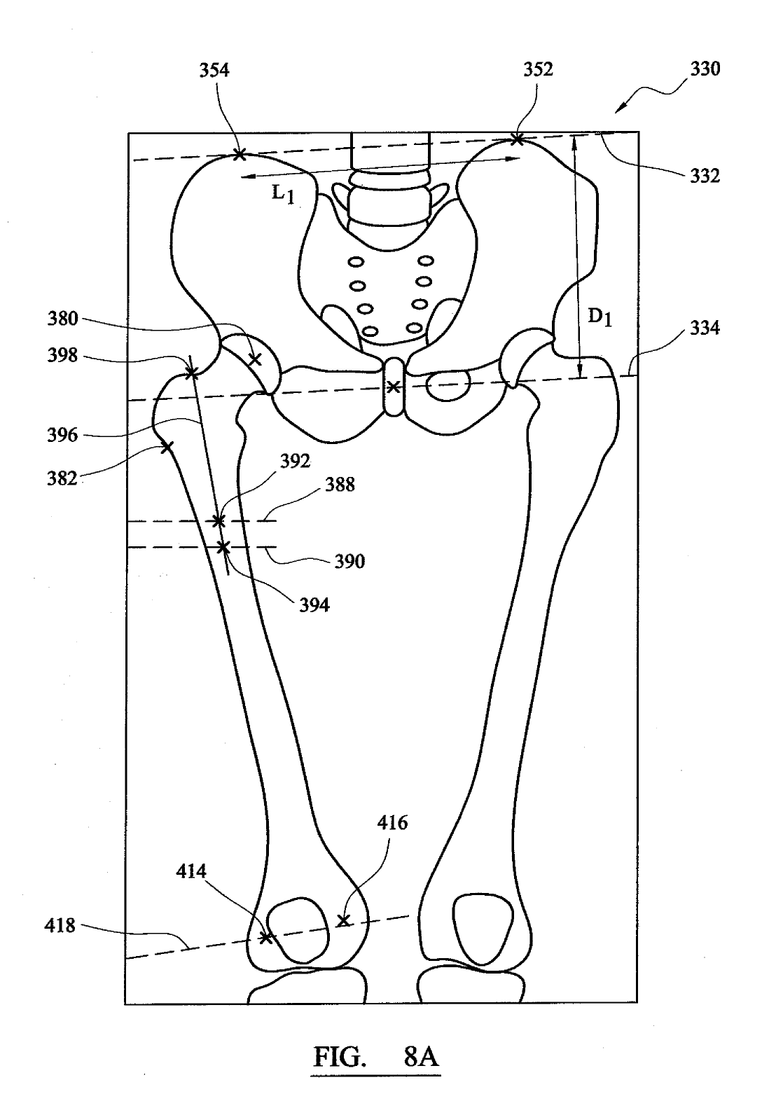

FIGS. 7A and 7B show a flow chart illustrating a method for determining the orientation of a pelvis and anatomical planes of the pelvis from images captured by the system shown in FIG. 5;

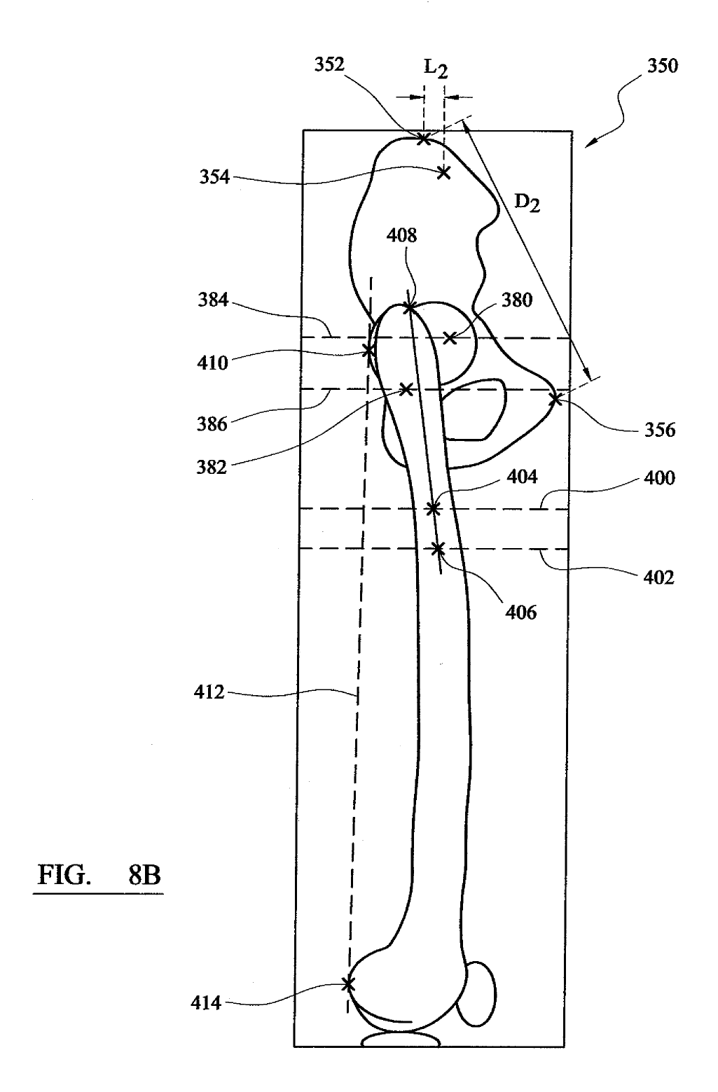

FIGS. 8A and 8B show schematic representations of X-ray images capture by the system shown in FIG. 5 and which are used in the processes of the method illustrated in FIGS. 7A & 7B;

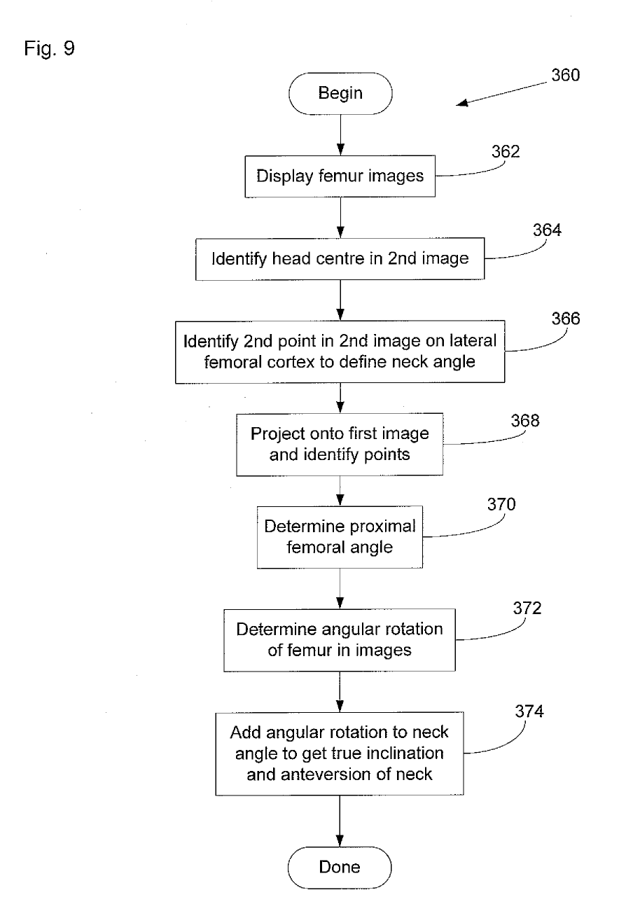

FIG. 9 shows a flow chart illustrating a method of analyzing the X-rays shown in FIGS. 8A and 8B to determine the orientation of the femoral neck;

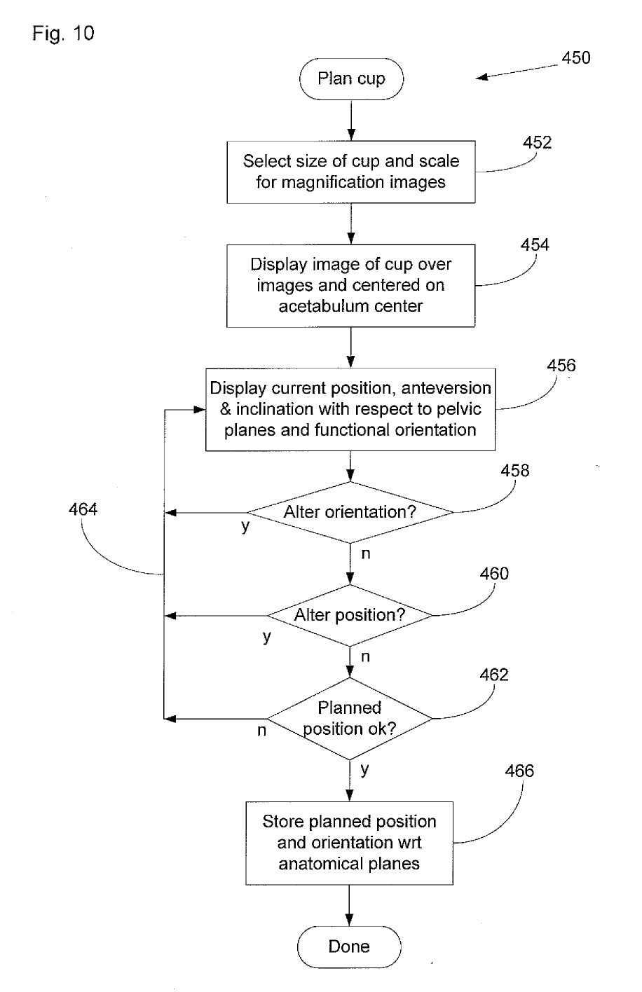

FIG. 10 shows a flow chart illustrating a method for planning an acetabular cup position;

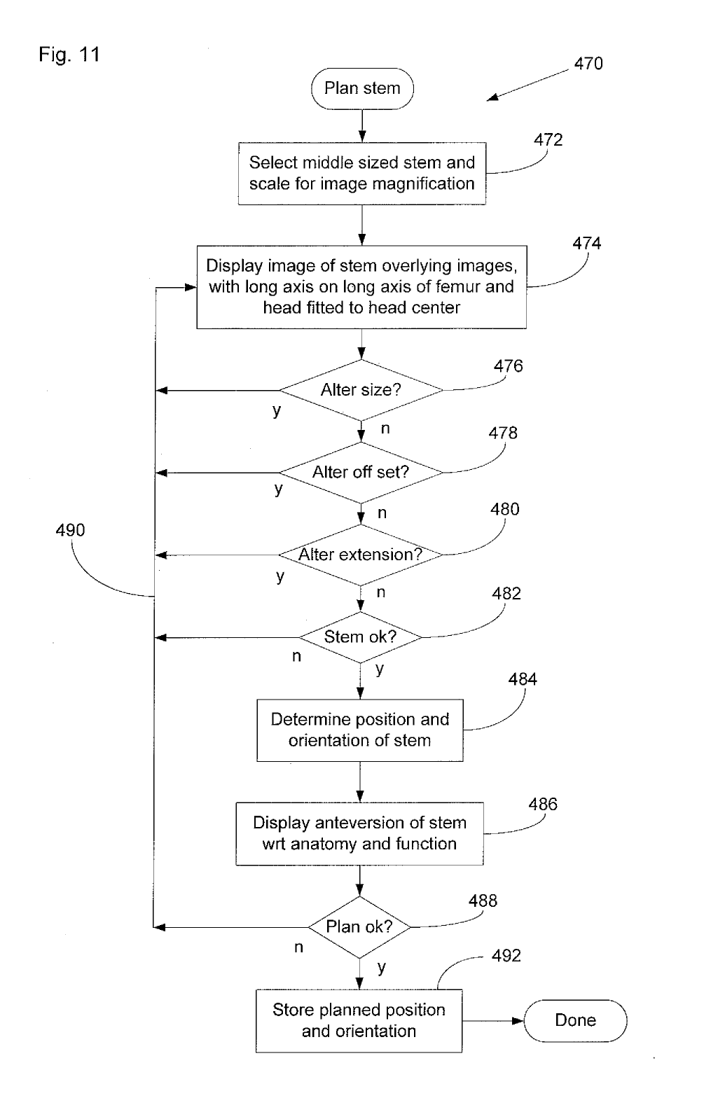

FIG. 11 shows a flow chart illustrating a method for planning a femoral implant position;

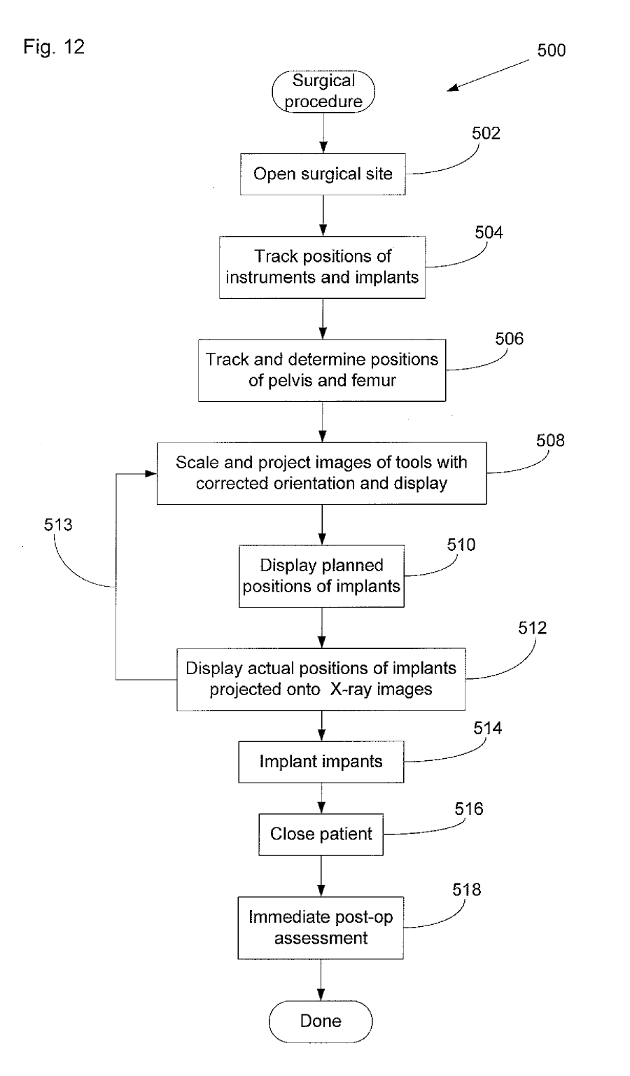

FIG. 12 shows a flow chart illustrating a method for carrying out a computer aided surgical procedure using the implant positions planned using the methods illustrated in FIGS. 10 and 11;

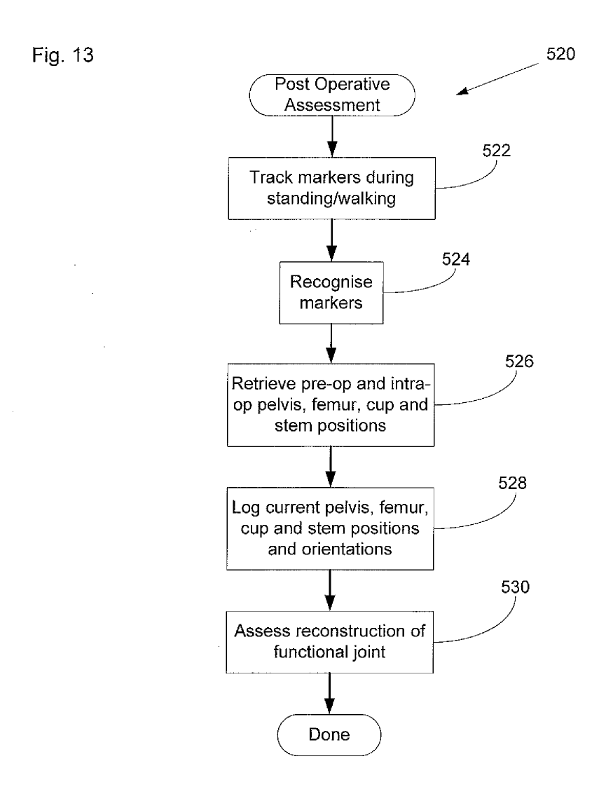

FIG. 13 shows a flow chart illustrating a method for carrying out a post operative assessment of the joint replacement; and

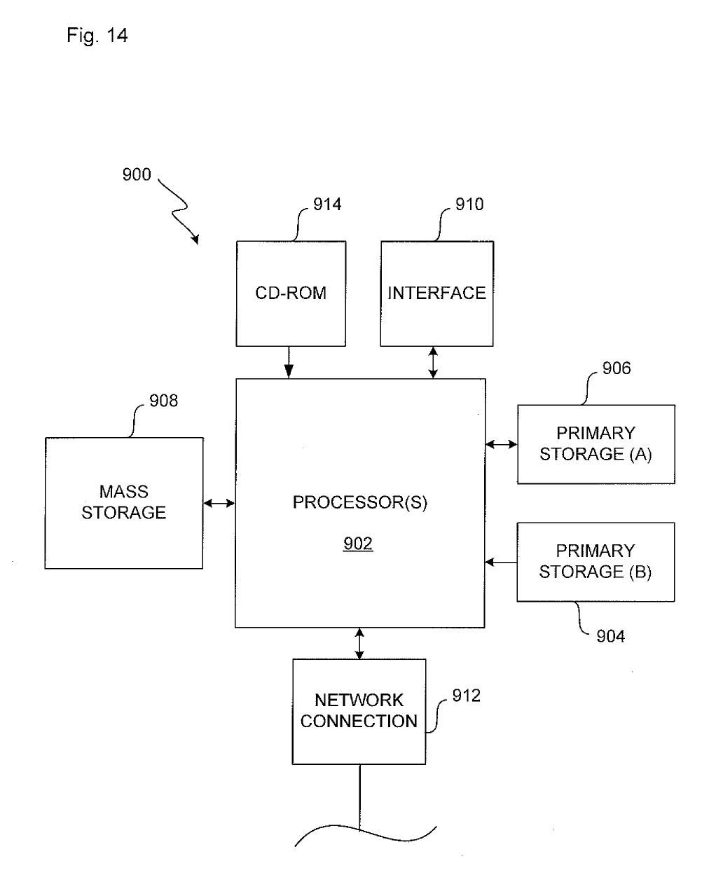

FIG. 14 shows a schematic block diagram of a data processing apparatus which can be configured by computer program code to provide some of the method aspects of the invention.

Similar items in different Figures share common reference numerals unless indicated otherwise.

Before describing an embodiment in detail an overview of the invention will be given. The overview and embodiment will be described in the context of hip replacement, but the invention is not limited to applicability in hip replacement and can be of use in relation to any joint, or part of a joint, and indeed to any body structures involving relatively movable parts or bones. For example, the invention can be applied to hip, knee, shoulder, ankle, wrist, elbow joints as well as in the spinal and cranial areas.

In the following orientation and direction will generally be used to refer to the angular properties of an entity, and position will generally be used to refer to the location properties of an entity, such as its co-ordinates in a frame of reference. The term configuration will generally be used to refer to the combination of the position and orientation of an entity or entities. Therefore, an entity's overall spatial attributes are a combination of its position and orientation. In a Cartesian frame of reference, an entity's position can be defined by its x, y and z co-ordinates and its orientation by three angular components, often referred to as yaw, roll and pitch. However, it will be appreciated that in practice only two independent angular components need to be specified in order to fully specify an entity's orientation.

The invention integrates an external reference direction into a measurement of the orientation of a body part, or body parts, made on a patient with the body part, or parts, in their functional configuration, e.g. a patient's hip while the patient is standing. This allows the functional orientation of the body part to be used as input data for the planning and execution of a surgical process using navigation.

Hence, the functional orientation of the body part, that is the orientation or configuration of the body part during normal use, can be considered as part of the planning of a procedure to correct or replace the body part, e.g. through a prosthetic hip replacement, so as to improve the performance of the body part, more accurately reproduce the prior performance of the body part and reduce post operative complications, such as implants becoming loose or failing.

Further, there are significant variations in functional orientation which are patient specific and can be of significant magnitude. Hence, by integrating the functional orientation information into the surgical plan on a patient by patient basis, the surgical procedure is bespoke for each patient.

FIG. 1 shows a schematic side view 100 of the pelvis 102 of a patient as the pelvis is being imaged onto an imaging plane 104. A marker 106 trackable by a tracking system is attached to the pelvis. Plane 108 corresponds to an anatomical plane of the pelvis, and in this instance corresponds to the frontal pelvic plane, which is defined by three anatomical points 110 on the pelvis. The image of the pelvis 112 is captured and associated with a reference direction 114. In this instance, the reference direction is the direction of the Earth's gravitational field and the reference direction is determined at the time the image 112 is captured by using a trackable marker 116. Hence, the image of the pelvis captured at the imaging plane 104, has encoded in it, or otherwise associated with it, information specifying the orientation of the pelvis, relative to the reference direction 114, as represented by angle 118. Angle 118 provides an off-set or correction angle between the anatomy of the pelvis and the functional direction of the pelvis. The corrected frontal plane of the pelvis that should be used for planning is plane 119. Hence, the functional angle of the pelvis, i.e. the angle of the pelvis in use, relative to a reference direction is known.

FIG. 2 shows a side view 120 of the same pelvis but with the patient recumbent on a table in an operating theatre. In this configuration the functional orientation of the pelvis is no longer known. The patient's anatomy is registered to the previously captured image. Planning can be carried out using the anatomical landmarks on the image 112 which has not been corrected for gravity. Then the surgical plan is corrected to recapture the functional orientation of the pelvis by introducing the off-set or correction angle into the surgical plan as illustrated by image 124.

For example, if in the functional state, the angle between the frontal plane of the pelvis and the direction of gravity is 35^{B}, then the plan made on the un-corrected image needs to have an angle of 35^{B} relative to the frontal plane added to the planned angular positions so that the functional orientation of the pelvis can be re-created in the operating theatre.

A number of approaches to including information specifying the orientational relationship between a reference direction, such as gravity, and a persons body part or parts in a functional state can be used. For example, the orientation of a person's bone relative to the direction of gravity can be measured directly while standing, or performing some other usual physical activity involving the bone. That orientational data can then be included in a surgical plan which will be executed when the patient is in another position, using a navigated surgical approach. A marker identifying the reference direction can be incorporated in an image data set to be used as input to planning a navigated procedure. This approach can include the using 2D image data sets to generate a 3D morphed model of the bone using statistical shape models. The direction of the reference direction can be included in a transformation matrix for an image data set so as to map the captured image data into the reference frame of a tracking system used during navigated surgery.

The functional orientation of the body parts can also be used in a pre-operative assessment of the patient, for example: to assess local and global stresses and pressure distributions; in passive and dynamic joint force balancing; establishing a preferred range of locations for implant positioning, preferred implant type and preferred treatment modality for specific patients related to their activity levels, age, cultural background and other patient specific factors.

With reference to FIG. 3 there is shown a flow chart illustrating at a high level a method 200 for carrying out a surgical procedure to more accurately reproduce the function of a body part, and in particular a hip implant. The method begins with an image capture step and any associated image processing 202. An image or images of the patient are captured with the patient's joint in a loaded or functional state in which the orthopaedic of mechanical behaviour of the joint in its typical working state is captured. The joint may be imaged in a static or dynamic state.

For example, for leg joints, the weight of the person may be the loading and images may be captured of the hip, knee or ankle joints with the person standing, walking, jumping, hopping, sitting, or moving between sitting and standing positions. For other joints, such as arm joints and spinal joints, the loading can again be the persons weight and can be supplemented, for example by carrying or holding a bag. Again images can be captured of joints in static and dynamic states.

Various different imaging modalities can be used to capture images of the joints in their functional state. For example, X-ray imaging, CT imaging, Magnetic Resonance (MR) imaging, ultrasound imaging, X-ray fluoroscopy can be used for capturing internal images of the joints. Still photography and video can also be used to capture images of naked patients from which orientational information about the functional state of the patient's joints can be derived. If an imaging technique cannot be used to capture images of the joint in a functional state, for example MR imaging, then the effect of gravity can be incorporated into the MR images by registering the MR images with an image of the joint which was captured in the functional state, e.g., by an X-ray imaging technique.

After the images have been captured, at step 204, the surgical joint replacement procedure is planned using the captured images and taking into account the relative positions and relative orientations of the components of the joints in their functional state. The surgical planning can initially be performed based on the functional orientation information in order to recreate the functional configuration of the original joint with the prosthetic components. The surgical plan can then be fine tuned based on a number of secondary considerations, such as: range of motion; impingement; load transfer; stress distribution; wear; cup fixation; stability; luxation; and other similar considerations in orthopaedic surgical planning.

Planning may be a pre or intra operative procedure or may include pre and intra operative elements. Once the surgical plan, which has now been corrected to take into account the functional configuration of the joints, has been completed, then at step 206, the surgical procedure is carried out using navigated instruments, tools and implants using a computer aided surgery (CAS) system. A CAS system generally includes some form of tracking system which can track the positions and orientations in a reference frame of the tracking system of suitably marked tools, instruments, implants and body parts. Images of the patients body parts and the planned position and orientation of the implants and instruments, together with the real time position and orientation of the actual implants and instruments, can be displayed to the surgeon so as to guide the surgeon in an Image Guided Surgery (IGS) approach.

Various different tracking technologies can be used, such as wired or wireless tracking, and various wireless tracking technologies can be used such as ultrasound based, electromagnetic radiation based, e.g. infra red and RF, and using passive or active markers. An example tracking technology is an infra red based tracking system provided by Brain LAB AG under the name Vector Vision. This approach uses marker arrays having three IR reflective balls which reflect IR radiation to two offset IR cameras which capture stereographic images of the balls from which the position of the marked item can be determined.

In one embodiment, the invention makes use of a wireless electromagnetic field based tracking and marker system. The tracking system generates a high frequency magnetic field having a characteristic distribution. The marker includes three mutually perpendicular sensor coils which can each measure a different component of the magnetic field distribution by induction. On board electronics wirelessly transmits data from the sensor coils, and a unique identifier for the maker, back to the tracking system which determines the position and orientation of the marker within the reference frame of the tracking system. Further details of a suitable marker and tracking system are disclosed in greater detail in U.S. patent publication no. US 2003/0120150 A1 (U.S. patent application Ser. No. 10/029,473) which is incorporated herein by reference in its entirety for all purposes.

After the surgery has been completed, there can be some immediate post operative assessment of the joint, e.g. to determine the range of motion, and then at step 208 the patient is allowed to recover, which may include physiotherapy. After patient recovery, post operative assessment of the surgical procedure can optionally be carried out at step 210, to assess both how well the prosthetic joint functions and also how well the prosthetic joint has recreated, or matches, the original joint in terms of its functional performance.

Various steps of the overall method will now be described in greater detail.

FIG. 4 shows a flow chart illustrating an image capturing method 220 corresponding generally to the image capturing step 202 of FIG. 3 in greater detail. FIG. 5 shows a schematic block diagram of an image capturing system 240 and FIG. 6 shows a schematic diagram of an X-ray cassette 270 for use in the system shown in FIG. 5. The image capturing system 240 includes a tracking system 242 which can track and determine the position (in terms of x, y and z co-ordinates) and orientation (in terms of pitch, yaw and roll angles, ψ, φ, θ) of suitably marked entities within the reference frame or co-ordinate system 244 of the tracking system. In FIG. 5, markers are represented by stars. The tracking system can be in communication with, or integrated, into a computer system 243 which can carry out data processing operations on data received from the tracking system, and which can also include surgical planning software as will be described in greater detail below. A patient 246 who is going to have hip replacement surgery has a first marker 248 attached to their femur and a second 250 marker attached to their pelvis. The system also includes a marked source of X-rays 252 and a marked X-ray film cassette 256 with which X-ray images of the patient's hip can be captured. An X-ray calibration phantom 258 can also be provided. The system also includes a device 260 for generating a reference direction. In it's the reference direction can correspond to the local direction of the Earth's gravitational field. The reference direction device, can in a simple embodiment simply be a plumb line or spirit level or similar which bears a marker so that the tracking system can determine the direction of the Earth's gravitational field therefrom. In one embodiment, the system the electromagnetic field based tracking system described above and the markers are each wireless magnetic field sensors. Hence at any time the tracking system can determine and record the position of any of the X-ray source, X-ray film, femur, pelvis and direction of gravity.

FIG. 6 shows an alternate embodiment 270 of the X-ray cassette shown in FIG. 5. The X-ray cassette 270 includes an X-ray sensitive film, or detector for digital imaging, and a marker 274 trackable by the tracking system is attached to the casing 276 of the X-ray cassette. A corner of the casing 276 includes an aperture in which a trackable marker 278 is freely suspended by a wire 280. Hence, cassette 270 has the reference direction device built into it and a separate reference direction device is not required.

Returning to FIG. 4, at step 222, markers 248, 250 are attached to the patient's pelvis and femur adjacent the hip joint and a marker is attached to the X-ray film 256. Then at step 224 at least one X-ray in the anterior-posterior direction is captured. Preferably two x-rays are captured from different directions approximately perpendicular to each other.

It is necessary to be able to determine the magnification of the X-ray system in order to determine the size of the patient's bones accurately. In order to determine the magnification it is necessary to know the position of the source of the X-rays. A trackable marker can be attached to the X-ray source. Alternatively, back projection from a calibration phantom can be used to calculate the position of the X-ray source. In one approach, as illustrated by step 226, the X-ray calibration phantom 258 is used and from the known size of the phantom and the size of the X-ray image of the phantom, the location of the source can be determined in a known manner. In another approach, as illustrated by step 228, a pre-calibration of the X-ray system is carried out in which the positions of the source and film are determined by the tracking system as an image of the phantom is captured. The position of the source can then be determined from the known size of the phantom and the size of the captured image of the phantom using singular value decomposition.

The position and orientation data of the X-ray source, film, pelvis and femur and the direction of gravity are determined by tracking the position and orientation of the markers as the X-ray images are captured and stored at step 230. If a digital X-ray imaging system is being used, then at step 232 the digital X-ray images are stored together with a patient ID at step 232. If X-ray film is being used, then the images derived from the X-ray film are scanned and digital images are stored together with a patient ID at step 234. Then at step 236, the magnification factor for each captured image is determined by the computer system 243 and stored in association with the saved images.

FIGS. 7A and 7B show a flow chart illustrating a first part of a method 300 for planning the position of a hip joint to be implanted which is corrected for the functional configuration of the hip joint, and corresponding generally to step 204 of FIG. 3. At step 302 the captured image of images of the hip joint are displayed by planning software running on computer system 243 to the user. FIGS. 8A and 8B respectively show captured X-ray images of the patient's pelvis from the front 330 in a generally anterior-posterior direction and from the side 350 in a generally lateral-medial direction.

At present, the computer system and planning software knows the direction of gravity in the reference frame of the tracking system and also the orientation of the X-ray film, and hence images, in the reference frame of the tracking system. Therefore the orientation of the images relative to the direction of gravity can be determined. The next general operation is to determine the orientation of the relevant parts of the patient's anatomy, in this case the femur and pelvis, relative to the images. As the orientation of the film relative to the direction of gravity is known, the orientation of the body parts relative to the direction of gravity can therefore be determined.

The planning software allows a user to identify points in the displayed images using a cursor and a pointing device, such as a mouse. Various anatomical points on the pelvis and femur are identified in the images and basic trigonometry is applied to determine the orientation of the femur and pelvis relative to the displayed images.

In greater detail, at step 304, the positions of the left and right anterior superior iliac spine 352, 354 and the pubic symphysis 356 are identified in the first image shown in FIG. 8B. The relative rotation of the patient between images can be determined from the orientation data for the pelvic marker. At step 306, using the relative rotation data, a line 332 passing through points 352 and 354 is projected onto the second image 330. Similarly a second line 334 parallel to line 332 and passing through point 356 is also projected onto the second image 330. Then the positions of the left and right anterior superior iliac spine 352, 354 and the pubic symphysis 356 are identified in the second image 330 at step 308.

Then at step 310, using the separations between the same points on different images, the orientation of the pelvis relative to the images can be determined. In greater detail the separation between the left and right anterior superior iliac spine, L₁, is determined from the second image, and the separation between the same two points, L₂, is determined from the first image. Similarly, the separation between the right anterior superior iliac spine and pubis symphysis, D₁, is determined from the second image, and the separation between the same two points, D₂, is determined from the first image. Then the magnification factor for each image is used to scale L₁, L₂, D₂ and D₂ to provide their actual distances. Then the angular relationship between the pelvis 330 and the second image is determined using θ_{L}=tan⁻¹(L₂/L₁) and φ_{D}=tan⁻¹(D₂/D₁), where the scaled real actual distances are used. Hence, θ_{L} and φ_{D} define the relative orientation between the pelvis and the images and hence the relative orientation between the pelvis and the direction of gravity can be determined.

Then a number of operations are carried out to determine the local anatomical geometry of the pelvis. These operations allow the cardinal or major anatomical planes of the pelvis to be identified. The frontal plane of the pelvis has already been defined by the left and right anterior superior iliac crest points and mid point of the symphysis pubis. Then at step 312, the positions of the left and right inferior ischeal tuberosities are identified in the first image and the line passing through those points is projected onto the second image. Then the positions of the same two points are identified on the line on the second image at step 314. Once the position of the two points has been determined for both the images, at step 316 the plane including the two points and being perpendicular to the frontal plane is calculated. This determines the transverse plane of the pelvis.

Then, a number of operations are carried out to identify the centre of the acetabulum and determine the position of the third anatomical plane of the pelvis. A target or template comprising cross hairs and a number of concentric circles is overlaid on the X-ray images and its position can be moved by the user. The user moves the target over the first image until it is considered to be centred on the acetabulum. The position of the centre of the acetabulum is determined and also the user can enter a command selecting a one of the concentric circles, which provide a number of graduations on a acetabular cup size scale, to be selected so that the approximate size of the acetabular cup is automatically determined. The line passing through the centre of the acetabulum is projected onto the second image 330 and the position centre of the acetabulum is identified on the second image at step 320. Then the position and orientation of the third pelvic plane is calculated using the constraints that the third plane is perpendicular to the frontal plane and transverse plane and also passes through the centre of the acetabulum. The third plane is the sagittal plane of the pelvis. The sagittal plane does not necessarily pass through the centre of the acetabulum and can be at any position in the pelvis while being perpendicular to the frontal and transverse planes.

Hence, now the orientation of the local anatomical geometry of the pelvis relative to the direction of gravity can be derived. Often the orientation of the acetabular cup is defined in terms of the angle of inclination and the angle of version, where the angle of inclination is defined relative to the transverse plane of the pelvis and the angle of version is defined relative to the sagittal plane of the pelvis. However, as the acetabular cup is a part of the pelvis, once one measure of the orientation of the pelvis relative to the direction of gravity is defined, the orientation of the acetabular cup relative to the direction of gravity is also defined.

The method continues by determining the orientation of the second component of the joint relative to the reference direction of the direction of gravity. With reference to FIG. 9 there is shown a flow chart illustrating a method 360 for determining the orientation of the femur relative to the first and second images 350, 330. The method begins and at step 362 the captured images 350,330 including the femur are displayed to the user by the planning software. The position of the centre of the femoral head 380 is identified on the second image 330 at step 364. The centre of the head may coincides generally with the centre of the acetabulum. Then at step 366 the direction of the longitudinal axis of the femoral neck is determined by identifying the position of a second point in the second image 330, for example a point 382 on the lateral femoral cortex. The lines 384, 386 passing through the two points defining the neck axis are projected onto the first 350 image at step 368 and the positions of the same two points are identified on the first image. Then using trigonometry, the direction of the femoral neck axis relative to the images is determined based on the lengths of the femoral neck axis in the different views and the known relative orientations of the different views in a manner similar to that described above for the pelvis.

Then at step 370, a number of operations are carried out to determine the orientation of the proximal part of the femur. Firstly, two levels 388, 390 approximately 100 mm below the femoral head centre are identified on the second image 330. Then the position of the central point of the inner cortex distance for each level 392, 394 is determined. Then the line 396 passing through those points is constructed and displayed on the image 330. The point 398 at which line 396 intersects the piriformis groove is identified in the image and its position determined. The lines 400, 402 passing through the upper and lower points 392, 394 are in the second image are projected onto the first image 350 and the positions of the mid point of the inner cortex 404, 406 for the upper and lower lines are identified and determined. Then the position of the lowest point 408 in the piriformis groove notch is identified and determined in the first image. Then the line most closely passing through point 408 and points 404 and 406 is determined. The orientation of this line specifies the orientation of the proximal axis of the femur.

Then at step 372 a number of operations are carried out to determine the rotation of the femur in the images relative to the pelvis so as to determine the orientation of the neck angle relative to the pelvis in the functional state. Firstly, the position of the posterior protrusion 410 of the lesser trochanter is identified in the first image 350 and determined. Then the position 414 of the tangential point of contact of a line 412 from point 410 and the most posterior condyle is identified in the first image and determined. Then a line perpendicular to line 412 and passing through point 414 is constructed and the position of the point 416 (not shown in FIG. 8B) where that line (also not shown in FIG. 8B) tangentially intersects the other condyle is identified and determined.

Then the line passing 418 through the two tangential condyle points is determined and projected onto the second image 330. Then the positions of the two points 414, 416 on the posterior of the condyles are identified in the second image 330. Then, using similar trigonometry to that described above, the angular rotation of the femur relative to the images is determined using the separation between points 414 and 416 in the first image and in the second image. Finally the true orientation of the femoral neck in 3D space is determined at step 374 from the angular rotation of the femur in the images and the neck angle determined previously at step 366. This provides the inclination and anteversion angles of the femoral neck relative to the images and hence the inclination and anteversion angles relative to the reference direction can be determined.

With reference to FIG. 10 there is shown a process flow chart illustrating a cup planning part 450 of a surgical planning process corresponding generally to step 204 of FIG. 3. The planning program begins and initially displays the X-ray images 330, 350 to a user. Then at step 452, a virtual acetabular cup implant having a size most closely matching the target size used previously to identify the centre of the acetabulum is selected from a range of real acetabular cup implants. An image of the selected virtual acetabular cup implant is scaled using the respective magnification factors for the images. Then at step 454, the image of the acetabular cup implant is displayed overlaid on both the X-ray images and centred on the acetabulum centre. The relative orientation between the two image is known and so the acetabular cup image shows the acetabular cup as viewed from the two different directions.

At step 456, the position of the acetabular cup is determined and displayed to the user, together with the anteversion and inclination angles of the cup relative to the pelvic planes and also relative to the reference direction. Default values can be used for the initial orientation, such as an inclination of 45^{E} inclination and 15^{E} anteversion relative to the reference direction. Hence, the orientation relative to the pelvic planes can be calculated as the orientation of the pelvis relative to the reference direction has already been determined.

At step 458, the user can enter commands to increase or decrease the inclination or anteversion of the cup and processing returns to step 456 at which the display is updated to show the changed orientation of the cup and also the changes in the inclination and anteversion angles relative to anatomy and function. At step 460, the user can enter commands to change the position of the cup and processing returns to step 456 at which the display is updated to show the changed position of the cup and also the distance of the centre of the cup from the centre of the acetabulum in the sagittal-inferior, anterior-posterior and medial-lateral directions. As represented by process flow return line 464, stages 458 and 460 can be repeated as many times as necessary and in any order until the user is happy with the planned cup position. At step 462, the user can observe the planned cup position and orientation and if the planned cup configuration is acceptable, then they can enter a command causing the plan for the acetabular cup implant to be stored at step 466. The position and orientation data is stored relative to the anatomical planes of the pelvis. Subsequently the navigation system uses the rigid body of pelvis as a reference frame as the navigation marker is attached to the pelvis during surgery and the pelvis anatomy is registered using the reference marker for navigation purposes. Hence, the plan is stored in the pelvis coordinate system. In an alternative embodiment, the plan can be stored in one reference system and then transformed to the pelvis coordinate system for use during navigated surgery.

With reference to FIG. 11 there is shown a process flow chart illustrating a femoral stem planning part 470 of the surgical planning process corresponding generally to step 204 of FIG. 3. The process 470 also displays the X-ray images to the user and at step 472 a virtual representation of a femoral stem implant having medium size is selected from a range of actual femoral stem implants. Images of the stem viewed from the appropriate directions are displayed overlaid on the X-ray images at step 474 and with the long axis of the stem on the proximal femoral axis and the prosthetic femoral head centred on the actual femoral head. Then at step 476, the user can enter a command to change the size of the stem and select a different sized stem for use in the planning process. At step 478, the user can enter a command to alter the off set of the femoral implant. At step 480, the user can enter a command to alter the extension of the femoral implant. At step 482, the user can enter a command indicating that the selected stem is acceptable as the basis of the surgical plan. As represented by process flow return line 490, any of steps 476 to 482 can be repeated any number of times and in any order so as to allow the appropriate stem to be selected.

Then at step 484, the position of the head and the orientation of the neck axis of the selected stem is determined and at step 486, the anteversion and inclination of the selected stem is displayed to the user with respect to the anatomy of the femur, i.e. the femoral axes, and with respect to function, i.e. the reference direction. If the planned position is determined to be acceptable at step 488, then at step 492 the planned position and orientation data for the stem are stored. Otherwise processing returns to step 474, or any preceding step, and the stem planning process can be repeated or fine tuned until an acceptable plan has been generated.

Although illustrated as separate and sequential processes it will be appreciated that processes 450 and 470 can be carried out in parallel or preferably combined into a single integrated planning process in which the positions and orientations of the cup and stem implants are planned together.

Hence, as the X-rays have been used in the planning process, the planning process has been based upon the functional configuration of the hip joint. Further, as the orientation of the hip joint relative to the reference direction is also known, by tracking the positions of the parts of the hip joint during navigated surgical positioning of the implants, the functional configuration of the hip can be more accurately recreated.

With reference to FIG. 12 there is shown a flowchart illustrating a computer aided surgical method 500 for carrying out a hip replacement procedure using the surgical plan derived from the above described method and generally corresponding to step 206 of FIG. 3. The surgical procedure is carried out using a computer aided surgery (“CAS”) system including a tracking system and including image guided surgery (“IGS”) software to provide visual guidance via a display device to assist the surgeon in accurately locating and orienting the implants.

At step 502 the surgical site is opened. If trackable markers were already implanted in the femur and pelvis at the image capturing stage, then further markers do not need to be attached to the pelvis and femur. If not, then trackable markers are attached to the pelvis and femur so that the position and orientation of the pelvis and femur in the reference frame of the tracking system can be determined. The instruments and implants used during the procedure are also marked so as to be trackable and at step 504, the instruments and implants are tracked and data representing the current positions and orientations are supplied to the IGS software. Similarly at step 506, the patient's pelvis and femur are tracked and data representing the current positions and orientations are supplied to the IGS software.

The X-ray images are displayed to the user by the IGS software. Using the tracked positions of the femur and pelvis the X-ray images are registered with actual positions of the pelvis and femur in the reference frame of the tracking system. A number of methods can be used to register the X-ray images with the positions of the pelvis and femur. In a preferred embodiment an automatic registration procedure is used. In this procedure, the markers that were used during X-ray image capture are retained in the femur and pelvis and are also imaged by the X-ray image capture. The positions of the pelvic and femoral markers in the reference frame of the tracking system in the operating theatre are determined. The images of the pelvic and the femoral markers in the X-ray images is then mapped onto the actual positions of the markers in the reference frame of the tracking system so as to register the X-ray images with the patient.

The tracking system also supplies data indicating the identity of each of the tracked items to the IGS software. The implants include trackable markers which are hermetically sealed in biocompatible materials so that they can be permanently implanted in the human body. At step 508, the IGS software generates images of the implants and instruments which are scaled using the respective magnification factors for the two X-ray images and images corresponding to the views of the instruments in the different directions of the X-ray images are overlaid on the X-ray images so that the surgeon is provided with a virtual representation of the positions of the instruments and implants relative to the X-ray images and hence femur and pelvis.

At step 510, using the planning data, graphical and visual indications of the planned positions and orientations of the implants are displayed to the surgeon. The displayed information can include the planned position and orientation of the implants relative to local anatomy and also relative to function. Then a virtual representation of the implants can be displayed overlaid on the X-ray images showing the current position of the implants relative to the images and hence body. The images of the implants are scaled to match the X-ray images using the magnification factors and are displayed in different views to reflect the different directions of the X-rays. As represented by process flow line 513, as the implants and instruments are manipulated by the surgeon, the display is updated to reflect the current actual position and orientation of the implants together with the planned positions and orientations of the implants.

Hence, the display may show a planned cup orientation of 42 inclination and 23 anteversion relative to the pelvic planes and 68 inclination 32 anteversion relative to function, i.e. the direction of gravity. The surgeon is provided with the real time actual inclination and anteversion angles of the acetabular cup implant and so can use the displayed information to guide the accurate placement of the cup and other surgical steps involved in locating the implant.

At step 514, the pelvic and femoral implants are eventually implanted and at step 516, the surgical site is closed. At step 518, some immediate post-operative assessment of the surgical procedure can be carried out, for example by assessing the range of motion of the recreated hip joint. Alternatively or additionally, the position and orientation of the pelvis and femur can be determined by tracking the markers and comparing the configuration of the femur and pelvis with their pre-operative configuration.

With reference to FIG. 13 there is shown a method 520 for post operatively assessing the recreation of the functional joint and corresponding generally to step 210 of FIG. 3. At step 522 a tracking system is used to track and determine the positions and orientations of markers attached to the femur and pelvis and also to the femoral and pelvic implants with the joint in the functional state for which the images were originally captured, e.g. with the patient standing, walking, sitting or standing. Also the orientation of the reference direction, e.g. of gravity, is determined at the same time. At step 524, the tracking system recognises the identities of the implanted markers and at step 526, a computer based assessment system retrieves stored patient data including the pre-operative and intra-operative positions and orientations of the pelvis, femur, implanted acetabular cup and implanted femoral stem. The tracking system determines and logs the current positions and orientations of the same patient's pelvis, femur and implants in the with the joint in the functional state. Then at step 530 the success in recreating or reconstructing the functional joint can be assessed.

In one embodiment this can include determining the relative orientation of the femur and the pelvis, and also the orientation of the pelvis or femur relative to gravity, both before and after the operation. Then the before and after data can be compared to provide an indication of how well the functional joint has been re-created. It will be appreciated that simply determining the orientation of the pelvis or femur relative to the reference direction does not necessarily tell you how well the joint has been recreated. Although post operatively the pelvis may have the same tilt relative to the direction of gravity, it can be important that post operatively the femur still has the same orientation relative to the pelvis that it had pre-operatively. Similarly, although the relative orientation of the pelvis and the femur may be similar post and pre-operatively, the functional joint may not have been properly recreated, unless the pelvis has generally the same tilt relative to the direction of gravity post-operatively that it had pre-operatively or the femur still has generally the same orientation relative to the direction of gravity post-operatively that it had pre-operatively. Hence information about both the absolute orientation of a one of the joint components and the relative orientation of the two joint components best characterises whether the functional joint has been recreated.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

FIG. 14 illustrates a typical computer system that, when appropriately configured or designed, can serve provide the planning, computer aided surgery, IGS and assessment apparatus of this invention. The computer system 900 includes any number of processors 902 (also referred to as central processing units, or CPUs) that are coupled to storage devices including primary storage 906 (typically a random access memory, or RAM), primary storage 904 (typically a read only memory, or ROM). CPU 902 may be of various types including microcontrollers and microprocessors such as programmable devices (e.g., CPLDs and FPGAs) and unprogrammable devices such as gate array ASICs or general purpose microprocessors. As is well known in the art, primary storage 904 acts to transfer data and instructions uni-directionally to the CPU and primary storage 906 is used typically to transfer data and instructions in a bi-directional manner. Both of these primary storage devices may include any suitable computer-readable media such as those described above. A mass storage device 908 is also coupled bi-directionally to CPU 902 and provides additional data storage capacity and may include any of the computer-readable media described above. Mass storage device 908 may be used to store programs, data and the like and is typically a secondary storage medium such as a hard disk. It will be appreciated that the information retained within the mass storage device 908, may, in appropriate cases, be incorporated in standard fashion as part of primary storage 906 as virtual memory. A specific mass storage device such as a CD-ROM 914 may also pass data uni-directionally to the CPU.

CPU 902 is also coupled to an interface 910 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 902 optionally may be coupled to an external device such as a database or a computer or telecommunications network using an external connection as shown generally at 912. With such a connection, it is contemplated that the CPU might receive information from the network, or might output information to the network in the course of performing the method steps described herein.

Although the above has generally described the present invention according to specific processes and apparatus, the present invention has a much broader range of applicability. In particular, aspects of the present invention is not limited to any particular kind of joint or surgical procedure and can be applied to virtually body structure having relatively moving parts. Thus, in some embodiments, the techniques of the present invention could be applied throughout orthopaedics and skeletal parts. One of ordinary skill in the art would recognize other variants, modifications and alternatives in light of the foregoing discussion.

1-23. (canceled)

24. A computer-implemented method for planning a surgical procedure to be carried out on a joint of a subject, the joint including a first component and a second component, the first and second components being relatively movable, the method comprising the steps of:
capturing joint data describing the configuration of the joint while the joint is in a functional state wherein the joint is undergoing the joint's usual load bearing functional behaviour;
analyzing the joint data to determine joint component orientation data specifying the relative orientation of the first and second components; and
planning the orientation of at least one prosthetic implant to be used in the surgical procedure to recreate the joint using the joint component orientation data, wherein the planned orientation of the prosthetic implant recreates the functional state of the joint.

25. The method of claim 24, wherein the capturing step comprises capturing joint data in at least one image of the joint in the functional state.

26. The method of claim 24, wherein the capturing step comprises capturing joint data by tracking the position and/or orientation of the joint components in the functional state.

27. The method of claim 25, wherein the analyzing step comprises determining the orientation of the joint components relative to the or each image.

28. The method of claim 24, further comprising the step of determining the orientation of a reference direction when the joint data is captured.

29. The method of claim 28, further comprising the step of determining the orientation of at least one of the joint components relative to the reference direction.

30. The method of claim 24, further comprising the step of determining the orientation of the first component relative to the local anatomy of the subject and determining the orientation of the second component relative to the local anatomy.

31. The method of claim 24, wherein the planning step comprises displaying at least one image of the joint.

32. The method of claim 24, wherein the planning step comprises displaying a visual indication of the orientation of the first component relative to a reference direction and a first local anatomical direction and displaying a visual indication of the orientation of the second component relative to the reference direction and a second local anatomical direction.

33. The method of claim 24, further comprising the step of displaying a visual indication of a planned position and orientation of the prosthetic implants relative to the joint together with a visual indication of the real time position and orientation of the prosthetic implants relative to the joint thereby guiding the implantation of the prosthetic implants so as to recreate the functional joint.

34. A computer-implemented method for assessing a surgical procedure carried out to replace a joint of a subject, the joint including a first component and a second component, the first and second components being relatively movable, the method comprising:
before surgery, capturing joint data describing the configuration of the joint while the joint is in a functional state wherein the joint is undergoing the joint's usual load-bearing functional behaviour and determining joint component orientation data specifying the relative orientation of the first and second components;
after surgery, capturing joint data describing the configuration of the joint while the joint is in the same functional state and determining joint component orientation data specifying the relative orientation of the first and second components; and
comparing the before surgery joint orientation data and after surgery joint orientation data to assess how well the joint has been recreated.

35. A system for planning a surgical procedure to be carried out on a joint of a subject, the joint including a first component and a second component, the first and second components being relatively movable, the system comprising:
at least one data processor configurable by computer program code instructions to:
capture joint data describing the configuration of the joint while the joint is in a functional state wherein the joint is undergoing the joint's usual load bearing functional behaviour;
analyze the joint data to determine joint component orientation data specifying the relative orientation of the first and second components; and
plan the orientation of prosthetic implants to be used in the surgical procedure to recreate the joint using the joint component orientation data, wherein the planned orientation of the prosthetic implants recreates the functional state of the joint.

36. The system of claim 35, further comprising:
an image-capturing device for capturing at least one image of the joint of the patient in the functional state; and
a tracking device for tracking and determining the position and orientation of markers attachable to the subject and/or parts of the system.

37. A data processing apparatus for guiding a computer-aided surgery procedure being carried out on a joint of a subject, the joint including a first component and a second component, the first and second components being relatively movable, the apparatus including a data processing device configurable by computer program code instructions to:
display a visual indication of a planned position and orientation of the prosthetic implants relative to the joint, wherein the planned position and orientation are derived from planning data created from joint component orientation data specifying the relative orientation of the first and second components which has been derived from captured joint data describing the configuration of the joint while the joint is in a functional state wherein the joint is undergoing the joint's usual load bearing functional behaviour;
display a visual indication of the real-time position and orientation of the prosthetic implants relative to the joint thereby guiding the implantation of the prosthetic implants so as to recreate the functional joint.

38. A data processing apparatus for assessing a surgical procedure carried out to replace a joint of a subject, the joint including a first component and a second component and the first and second components being relatively movable, the apparatus comprising a data processing device configurable by computer program code instructions to:
obtain pre-surgery joint component orientation data specifying the relative orientation of the first and second components derived from captured joint data describing the configuration of the joint while the joint is in a functional state wherein the joint is undergoing the joint's usual load bearing functional behaviour,
obtaining post-surgery joint component orientation data specifying the relative orientation of the first and second components derived from captured joint data describing the configuration of the joint while the joint is in the same functional state; and
outputting comparison of the pre-surgery joint orientation data and post-surgery joint orientation data to allow how well the joint has been recreated to be assessed.
